(19) United States
(12) Patent Application Publication
(10) Pub. No.: US 2010/0330186 A1
(43) Pub. Date: Dec. 30, 2010

(54) MEDICAMENTS FOR INHALATION COMPRISING AN ANTICHOLINERGIC AND A BETAMIMETIC

(75) Inventors: Christopher John Montague MEADE, Maselheim (DE); Michel PAIRET, Biberach (DE); Michael P. PIEPER, Biberach (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERNATIONAL GMBH, Ingelheim am Rhein (DE)

(21) Appl. No.: 12/875,601

(22) Filed: Sep. 3, 2010

Related U.S. Application Data
(63) Continuation of application No. 10/891,552, filed on Jul. 15, 2004.
(60) Provisional application No. 60/507,982, filed on Oct. 2, 2003.

(30) Foreign Application Priority Data
Jul. 29, 2003 (EP) 03 017 163.1

(51) Int. Cl. A61K 9/12; A61K 31/4375; A61K 9/14; A61P 11/00; A61P 11/06

(52) U.S. Cl. 424/489; 514/305; 424/43; 424/46

(57) ABSTRACT

A pharmaceutical composition comprising a compound of formula 1

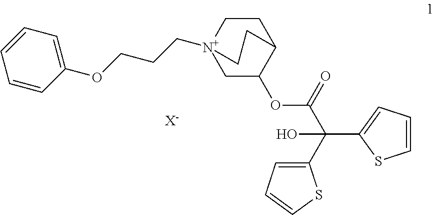

wherein X⁻ is an anion with a single negative charge, and a betamimetic, optionally together with a pharmaceutically acceptable excipient, the compound of formula 1 and the betamimetic optionally in the form of their enantiomers, mixtures of their enantiomers, their racemates, their solvates, or their hydrates, processes for preparing them, and their use in the treatment of respiratory tract diseases.

### RELATED APPLICATIONS

This application claims benefit of U.S. Ser. No. 60/507,982, filed Oct. 2, 2003, and claims priority to European Application No. 03 017 163.1, filed Jul. 29, 2003, each of which is hereby incorporated by reference in its entirety.

### FIELD OF THE INVENTION

The present invention relates to novel pharmaceutical compositions based on β₂ agonists and salts of an anticholinergic, processes for preparing them, and their use in the treatment of respiratory complaints.

### DESCRIPTION OF THE FIGURE

FIG. 1 shows an inhaler that may be used for administering the pharmaceutical combination according to the invention in inhalettes.

### DESCRIPTION OF THE INVENTION

The present invention relates to novel pharmaceutical compositions based on β₂ agonists and salts of a new anticholinergic 1, processes for preparing them and their use in the treatment of respiratory complaints. Such anticholinergics are disclosed in U.S. Patent Application Pub. No. 2003/0055080, which is hereby incorporated by reference.

Within the scope of the present invention the anticholinergic agents used are the salts of formula 1

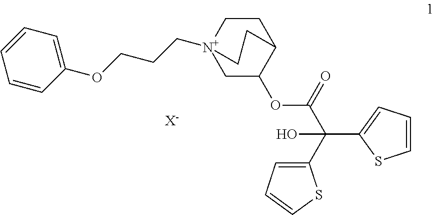

wherein X⁻ denotes an anion with a single negative charge, preferably an anion selected from the group consisting of fluoride, chloride, bromide, iodide, sulfate, phosphate, methanesulfonate, nitrate, maleate, acetate, citrate, fumarate, tartrate, oxalate, succinate, benzoate, and p-toluenesulfonate, optionally in the form of the racemates, the enantiomers, and the hydrates thereof.

Preferably, the salts of formula 1 are used wherein X⁻ denotes an anion with a single negative charge selected from among the fluoride, chloride, bromide, 4-toluenesulfonate, and methanesulfonate, preferably bromide, optionally in the form of the racemates, the enantiomers, and the hydrates thereof.

Most preferably, the salts of formula 1 are used wherein X⁻ denotes an anion with a single negative charge selected from among the chloride, bromide, and methanesulfonate, preferably bromide, optionally in the form of the racemates, the enantiomers, and the hydrates thereof.

Particularly preferred according to the invention is the salt of formula 1 wherein X⁻ denotes bromide.

Of particular interest according to the invention are the enantiomers of formula 1-en

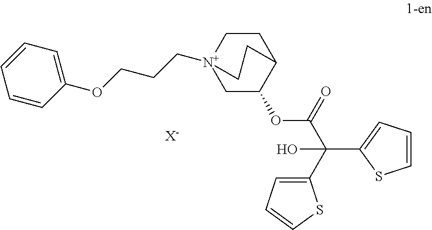

wherein X⁻ may have the meanings mentioned hereinbefore.

Surprisingly, an unexpectedly beneficial therapeutic effect can be observed in the treatment of inflammatory and/or obstructive diseases of the respiratory tract if the anticholinergic of formula 1 is used with one or more betamimetics 2.

The beneficial therapeutic effect mentioned above may be observed both when the two active substances are administered simultaneously in a single active substance formulation and when they are administered successively in separate formulations. According to the invention, it is preferable to administer the two active substance ingredients simultaneously in a single formulation.

According to the instant invention, preferred β₂ agonists 2 in the combinations according to the invention are selected from the group consisting of albuterol, bambuterol, bitolterol, broxaterol, carbuterol, clenbuterol, fenoterol, formoterol, hexoprenaline, ibuterol, isoetharine, isoprenaline, levosalbutamol, mabuterol, meluadrine, metaproterenol, orciprenaline, pirbuterol, procaterol, reproterol, rimiterol, ritodrine, salmeterol, salmefamol, soterenot, sulfonterol, tiaramide, terbutaline, tolubuterol, CHF-1035, HOKU-81, KUL-1248, 3-(4-{6-[2-hydroxy-2-(4-hydroxy-3-hydroxymethylphenyl)ethylamino]hexyloxy}butyl)benzenesulfoneamide, 5-[2-(5,6-diethylindan-2-ylamino)-1-hydroxyethyl]-8-hydroxy-1H-quinolin-2-one, 4-hydroxy-7-[2-{[2-{[3-(2-phenylethoxy)propyl]sulfonyl}ethyl]amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzylamino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-one, 1-(4-amino-3-chloro-5-trifluoromethylphenyl)-2-tert-butylamino)ethanol, and 1-(4-ethoxycarbonylamino-3-cyano-5-fluorophenyl)-2-(tert-butylamino)ethanol, optionally in the form of the racemates, the enantiomers, the diastereomers, and optionally the pharmacologically acceptable acid addition salts and the hydrates thereof.

According to the instant invention, more preferred β₂ agonists 2 are selected from the group consisting of bambuterol, bitolterol, carbuterol, clenbuterol, fenoterol, formoterol, hexoprenaline, ibuterol, pirbuterol, procaterol, reproterol, salmeterol, sulfonterol, terbutaline, tolubuterol, 3-(4-{6-[2-hydroxy-2-(4-hydroxy-3-hydroxymethylphenyl)ethylamino]hexyloxy}butyl)benzenesulfoneamide, 5-[2-(5,6-diethylindan-2-ylamino)-1-hydroxyethyl]-8-hydroxy-1H-quinolin-2-one, 4-hydroxy-7-[2-{[2-{[3-(2-phenylethoxy)propyl]sulfonyl}ethyl]amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzylamino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-one, 1-(4-amino-3-chloro-5-trifluoromethylphenyl)-2-tert-butylamino)ethanol, and 1-(4-ethoxycarbonylamino-3-cyano-5-fluorophenyl)-2-(tert-butylamino)ethanol, optionally in the form of the racemates, the enantiomers, the diastereomers, and optionally the pharmacologically acceptable acid addition salts and the hydrates thereof.

More preferably, the betamimetics 2 used as within the compositions according to the invention are selected from fenoterol, formoterol, salmeterol, 3-(4-{6-[2-hydroxy-2-(4-hydroxy-3-hydroxymethylphenyl)ethylamino]hexyloxy}butyl)benzenesulfoneamide, 5-[2-(5,6-diethylindan-2-ylamino)-1-hydroxyethyl]-8-hydroxy-1H-quinolin-2-one, 1-[3-(4-methoxybenzylamino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, and 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, optionally in the form of the racemates, the enantiomers, the diastereomers, and optionally the pharmacologically acceptable acid addition salts thereof, and the hydrates thereof. Of the betamimetics mentioned above, the compounds formoterol, salmeterol, 3-(4-{6-[2-hydroxy-2-(4-hydroxy-3-hydroxymethylphenyl)ethylamino]hexyloxy}butyl)benzenesulfoneamide, and 5-[2-(5,6-diethylindan-2-ylamino)-1-hydroxyethyl]-8-hydroxy-1H-quinolin-2-one are particularly preferred, optionally in the form of the racemates, the enantiomers, the diastereomers, and optionally the pharmacologically acceptable acid addition salts thereof, and the hydrates thereof. Of the betamimetics mentioned above, the compounds formoterol and salmeterol are particularly preferred, optionally in the form of the racemates, the enantiomers, the diastereomers, and optionally the pharmacologically acceptable acid addition salts thereof, and the hydrates thereof.

Examples of pharmacologically acceptable acid addition salts of the betamimetics 2 according to the invention are the pharmaceutically acceptable salts which are selected from among the salts of hydrochloric acid, hydrobromic acid, sulfuric acid, phosphoric acid, methanesulfonic acid, acetic acid, fumaric acid, succinic acid, lactic acid, citric acid, tartaric acid, 1-hydroxy-2-naphthalenecarboxylic acid, 4-phenylcinnamic acid, 5-(2,4-difluorophenyl)salicylic acid, or maleic acid. If desired, mixtures of the abovementioned acids may also be used to prepare the salts 2.

According to the invention, the salts of the betamimetics 2 selected from among the hydrochloride, hydrobromide, sulfate, phosphate, fumarate, methanesulfonate, 4-phenylcinnamate, 5-(2,4-difluorophenyl)salicylate, maleate, and xinafoate are preferred. Particularly preferred are the salts of 2 in the case of salmeterol selected from among the hydrochloride, sulfate, 4-phenylcinnamate, 5-(2,4-difluorophenyl)salicylate, and xinafoate, of which the 4-phenylcinnamate, 5-(2,4-difluorophenyl)salicylate and especially xinafoate are particularly important. Particularly preferred are the salts of 2 in the case of formoterol selected from the hydrochloride, sulfate, and fumarate, of which the hydrochloride and fumarate are particularly preferred. Of exceptional importance according to the invention is formoterol fumarate.

Salts of salmeterol, formoterol, 3-(4-{6-[2-hydroxy-2-(4-hydroxy-3-hydroxymethylphenyl)ethylamino]hexyloxy}butyl)benzenesulfoneamide, and 5-[2-(5,6-diethylindan-2-ylamino)-1-hydroxyethyl]-8-hydroxy-1H-quinolin-2-one, are preferably used as the betamimetics 2 according to the invention. Of particular importance according to the invention are salmeterol and formoterol salts. Any reference to the term betamimetics 2 also includes a reference to the relevant enantiomers or mixtures thereof.

In the pharmaceutical compositions according to the invention, the compounds 2 may be present in the form of their racemates, enantiomers or mixtures thereof. The separation of the enantiomers from the racemates may be carried out using methods known in the art (e.g., by chromatography on chiral phases, etc.). If the compounds 2 are used in the form of their enantiomers, it is particularly preferable to use the enantiomers in the R configuration at the C—OH group.

As an example, any reference to the most preferred compounds 2 according to the invention, the salts of salmeterol and formoterol, also includes the relevant enantiomeric salts of R-salmeterol, S-salmeterol, R,R-formoterol, S,S-formoterol, R,S-formoterol, S,R-formoterol, and the mixtures thereof, while the enantiomeric salts of R-salmeterol and R,R-formoterol are of particular importance. The compounds 2 may also be present according to the invention in the form of the hydrates or solvates thereof.

Where the present invention refers to betamimetics which are not in the form of salts, this is indicated by a reference to compounds 2′. For example, the preferred betamimetics 2′ according to the invention which are not in salt form include the free base of formoterol, salmeterol whereas the particularly preferred compounds 2 according to the invention are salmeterol xinafoate or formoterol fumarate.

Within the scope of the present invention the betamimetics 2 may possibly also be referred to as sympathomimetics or beta₂-agonists (β₂-agonists). All these terms are to be regarded as interchangeable for the purposes of the present invention.

In one aspect, the present invention relates to the abovementioned pharmaceutical compositions which contain, in addition to therapeutically effective quantities of 1 and 2, a pharmaceutically acceptable carrier. In another aspect, the present invention relates to the abovementioned pharmaceutical compositions which do not contain any pharmaceutically acceptable carrier in addition to therapeutically effective quantities of 1 and 2.

The present invention also relates to the use of therapeutically effective quantities of the salts 1 for preparing a pharmaceutical composition containing betamimetics 2 for treating inflammatory or obstructive diseases of the respiratory tract. Preferably, the present invention relates to the abovementioned use for preparing a pharmaceutical composition for treating asthma or COPD.

Within the scope of the present invention the compounds 1 and 2 may be administered simultaneously or successively, while it is preferable according to the invention to administer compounds 1 and 2 simultaneously.

The present invention further relates to the use of therapeutically effect amounts of salts 1 and betamimetics 2 for treating inflammatory or obstructive respiratory complaints, particularly asthma or COPD.

The proportions in which the active substances 1 and 2 may be used in the active substance combinations according to the invention are variable. Active substances 1 and 2 may possibly be present in the form of their solvates or hydrates. Depending on the choice of the compounds 1 and 2, the weight ratios which may be used within the scope of the present invention vary on the basis of the different molecular weights of the various salt forms. The pharmaceutical combinations according to the invention may contain 1 and 2′ generally in ratios by weight ranging from 1:5 to 500:1, preferably from 1:10 to 400:1.

The weight ratios specified below were based on the bromide of 1 and the free bases 2′ of the betamimetics salmeterol and formoterol which are particularly preferred according to the invention.

The pharmaceutical combinations according to the invention may contain the bromide of 1 and 2′ in the case of formoterol, for example, in ratios by weight ranging from 1:10 to 300:1, preferably from 1:5 to 200:1, preferably 1:3 to 150:1, more preferably from 1:2 to 100:1.

For example, without restricting the scope of the invention thereto, preferred combinations of 1 and 2 according to the invention may contain the bromide of 1 and formoterol 2′ in the following weight ratios: 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, and 100:1.

The pharmaceutical compositions according to the invention containing the combinations of 1 and 2 are normally administered so that the bromide of 1 and formoterol 2′ are present together in doses of 5 μg to 5000 μg, preferably from 10 μg to 2000 μg, more preferably from 15 μg to 1000 μg, better still from 20 μg to 800 μg.

For example, combinations of 1 and 2 according to the invention contain a quantity of the bromide of 1 and formoterol 2′ such that the total dosage per single dose is about 10 μg, 15 μg, 20 μg, 25 μg, 30 μg, 35 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg, 1000 μg, 1005 μg, 1010 μg, 1015 μg, 1020 μg, 1025 μg, 1030 μg, 1035 μg, 1040 μg, 1045 μg, 1050 μg, 1055 μg, 1060 μg, 1065 μg, 1070 μg, 1075 μg, 1080 μg, 1085 μg, 1090 μg, 1095 μg, 1100 μg, or similar It is clear to anyone skilled in the art that the suggested dosages per single dose specified above are not to be regarded as being limited to the numerical values actually stated. Fluctuations of about ±2.5 μg, particularly in the decimal range, are also included, as will be apparent to one of skill in the art. In these dosage ranges, the active substances 1 and 2′ may be present in the weight ratios given above.

For example, without restricting the scope of the invention thereto, the combinations in which the bromide is used as the salt 1 and in which 2 denotes formoterol fumarate is used as a preferred combination of 1 and 2, the pharmaceutical compositions according to the invention may contain for instance the following quantities for each single dose: 20 μg of 1 and 2.9 μg of 2, 20 μg of 1 and 5.7 μg of 2, 20 μg of 1 and 11.5 μg of 2, 20 μg of 1 and 17.2 μg of 2, 20 μg of 1 and 22.9 μg of 2, 20 μg of 1 and 28.5 μg of 2, 40 μg of 1 and 2.9 μg of 2, 40 μg of 1 and 5.7 μg of 2, 40 μg of 1 and 11.5 μg of 2, 40 μg of 1 and 17.2 μg of 2, 40 μg of 1 and 22.9 μg of 2, 40 μg of 1 and 28.5 μg of 2, 60 μg of 1 and 2.9 μg of 2, 60 μg of 1 and 5.7 μg of 2, 60 μg of 1 and 11.5 μg of 2, 60 μg of 1 and 17.2 μg of 2, 60 μg of and 22.9 μg of 2, 60 μg of 1 and 28.5 μg of 2, 100 μg of 1 and 2.9 μg of 2, 100 μg of 1 and 5.7 μg of 2, 100 μg of 1 and 11.5 μg of 2, 100 μg of 1 and 17.2 μg of 2, 100 μg of 1 and 22.9 μg of 2, 100 μg of 1 and 28.5 μg of 2, 200 μg of 1 and 2.9 μg of 2, 200 μg of 1 and 5.7 μg of 2, 200 μg of 1 and 11.5 μg of 2, 200 μg of 1 and 17.2 μg of 2, 200 μg of 1 and 22.9 μg of 2, 200 μg of 1 and 28.5 μg of 2, 300 μg of 1 and 2.9 μg of 2, 300 μg of 1 and 5.7 μg of 2, 300 μg of 1 and 11.5 μg of 2, 300 μg of 1 and 17.2 μg of 2, 300 μg of 1 and 22.9 μg of 2, 300 μg of 1 and 28.5 μg of 2, 400 μg of 1 and 2.9 μg of 2, 400 μg of 1 and 5.7 μg of 2, 400 μg of 1 and 11.5 μg of 2, 400 μg of 1 and 17.2 μg of 2, 400 μg of 1 and 22.9 μg of 2, 400 μg of 1 and 28.5 μg of 2, 500 μg of 1 and 2.9 μg of 2, 500 μg of 1 and 5.7 μg of 2, 500 μg of 1 and 11.5 μg of 2, 500 μg of 1 and 17.2 μg of 2, 500 μg of 1 and 22.9 μg of 2, 500 μg of 1 and 28.5 μg of 2, 600 μg of 1 and 2.9 μg of 2, 600 μg of 1 and 5.7 μg of 2, 600 μg of 1 and 11.5 μg of 2, 600 μg of 1 and 17.2 μg of 2, 600 μg of 1 and 22.9 μg of 2, 600 μg of 1 and 28.5 μg of 2, 700 μg of 1 and 2.9 μg of 2, 700 μg of 1 and 5.7 μg of 2, 700 μg of 1 and 11.5 μg of 2, 700 μg of 1 and 17.2 μg of 2, 700 μg of 1 and 22.9 μg of 2, 700 μg of 1 and 28.5 μg of 2, 800 μg of 1 and 2.9 μg of 2, 800 μg of 1 and 5.7 μg of 2, 800 μg of 1 and 11.5 μg of 2, 800 μg of 1 and 17.2 μg of 2, 800 μg of 1 and 22.9 μg of 2, 800 μg of 1 and 28.5 μg of 2, 900 μg of 1 and 2.9 μg of 2, 900 μg of 1 and 5.7 μg of 2, 900 μg of 1 and 11.5 μg of 2, 900 μg of 1 and 17.2 μg of 2, 900 μg of 1 and 22.9 μg of 2, 900 μg of 1 and 28.5 μg of 2, 1000 μg of 1 and 2.9 μg of 2, 1000 μg of 1 and 5.7 μg of 2, 1000 μg of 1 and 11.5 μg of 2, 1000 μg of 1 and 17.2 μg of 2, 1000 μg of 1 and 22.9 μg of 2, 1000 μg of 1 and 28.5 μg of 2.

For example, the active substance combinations according to the invention may contain the bromide 1 and 2′, in the case of salmeterol, in ratios by weight in the range from about 1:30 to 400:1, preferably 1:25 to 200:1, preferably 1:20 to 100:1, more preferably from 1:15 to 50:1.

For example, without restricting the scope of the invention thereto, preferred combinations of 1 and 2 according to the invention may contain the bromide of formula 1 and salmeterol 2′ in the following ratios by weight: 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1.

The pharmaceutical compositions according to the invention containing the combinations of 1 and 2 are usually administered so that the bromide of formula 1 and salmeterol 2′ are present together in dosages of 5 μg to 5000 μg, preferably from 10 μg to 2000 μg, more preferably from 15 to 1000 μg, even more preferably from 20 μg to 800 μg per single dose.

For example, combinations of 1 and 2 according to the invention contain an amount of the bromide of formula 1 and salmeterol 2′ such that the total dosage per single dose is about 15 μg, 20 μg, 25 μg, 30 μg, 35 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg, 1000 μg, 1005 μg, 1010 μg, 1015 μg, 1020 μg, 1025 μg, 1030 μg, 1035 μg, 1040 μg, 1045 μg, 1050 μg, 1055 μg, 1060 μg, 1065 μg, 1070 μg, 1075 μg, 1080 μg, 1085 μg, 1090 μg, 1095 μg, 1100 μg, 1105 μg, 1110 μg, 1115 μg, 1120 μg, 1125 μg, 1130 μg, 1135 μg, 1140 μg, 1145 μg, 1150 μg, 1155 μg, 1160 μg, 1165 μg, 1170 μg, 1175 μg, 1180 μg, 1185 μg, 1190 μg, 1195 μg, 1200 μg, 1205 μg, 1210 μg, 1215 μg, 1220 μg, 1225 μg, 1230 μg, 1235 μg, 1240 μg, 1245 μg, 1250 μg, 1255 μg, 1260 μg, 1265 μg, 1270 μg, 1275 μg, 1280 μg, 1285 μg, 1290 μg, 1295 μg, 1300 μg, 1305 μg, 1310 μg, 1315 μg, 1320 μg, 1325 μg, 1330 μg, 1335 μg, 1340 μg, 1345 μg, 1350 μg, 1355 μg, 1360 μg, 1365 μg, 1370 μg, 1375 μg, 1380 μg, 1385 μg, 1390 μg, 1395 μg, 1400 μg or similar. It is clear to anyone skilled in the art that the suggested dosages per single dose specified above are not to be regarded as being limited to the numerical values actually stated. Fluctuations of about ±2.5 μg, particularly in the decimal range, are also included, as will be apparent to the skilled man. In these dosage ranges, the active substances 1 and 2′ may be present in the weight ratios given above.

For example, without restricting the scope of the invention thereto, the combinations in which the bromide is used as the salt 1 and in which 2 denotes salmeterol xinafoate is used as a preferred combination of 1 and 2, the pharmaceutical compositions according to the invention may contain for instance the following quantities for each single dose: 20 μg of 1 and 18.2 μg of 2, 20 μg of 1 and 36.3 μg of 2, 20 μg of 1 and 72.6 μg of 2, 20 μg of 1 and 108.9 μg of 2, 20 μg of 1 and 145.2 μg of 2, 20 μg of 1 and 290.4 μg of 2, 40 μg of 1 and 18.2 μg of 2, 40 μg of 1 and 36.3 μg of 2, 40 μg of 1 and 72.6 μg of 2, 40 μg of 1 and 108.9 μg of 2, 40 μg of 1 and 145.2 μg of 2, 40 μg of 1 and 290.4 μg of 2, 60 μg of 1 and 18.2 μg of 2, 60 μg of 1 and 36.3 μg of 2, 60 μg of 1 and 72.6 μg of 2, 60 μg of 1 and 108.9 μg of 2, 60 μg of 1 and 145.2 μg of 2, 60 μg of 1 and 290.4 μg of 2, 100 μg of 1 and 18.2 μg of 2, 100 μg of 1 and 36.3 μg of 2, 100 μg of 1 and 72.6 μg of 2, 100 μg of 1 and 108.9 μg of 2, 100 μg of 1 and 145.2 μg of 2, 100 μg of 1 and 290.4 μg of 2, 200 μg of 1 and 18.2 μg of 2, 200 μg of 1 and 36.3 μg of 2, 200 μg of 1 and 72.6 μg of 2, 200 μg of 1 and 108.9 μg of 2, 200 μg of 1 and 145.2 μg of 2, 200 μg of 1 and 290.4 μg of 2, 300 μg of 1 and 18.2 μg of 2, 300 μg of 1 and 36.3 μg of 2, 300 μg of 1 and 72.6 μg of 2, 300 μg of 1 and 108.9 μg of 2, 300 μg of 1 and 145.2 μg of 2, 300 μg of 1 and 290.4 μg of 2, 400 μg of 1 and 18.2 μg of 2, 400 μg of 1 and 36.3 μg of 2, 400 μg of 1 and 72.6 μg of 2, 400 μg of 1 and 108.9 μg of 2, 400 μg of 1 and 145.2 μg of 2, 400 μg of 1 and 290.4 μg of 2, 500 μg of 1 and 18.2 μg of 2, 500 μg of 1 and 36.3 μg of 2, 500 μg of 1 and 72.6 μg of 2, 500 μg of 1 and 108.9 μg of 2, 500 μg of 1 and 145.2 μg of 2, 500 μg of 1 and 290.4 μg of 2, 600 μg of 1 and 18.2 μg of 2, 600 μg of 1 and 36.3 μg of 2, 600 μg of 1 and 72.6 μg of 2, 600 μg of 1 and 108.9 μg of 2, 600 μg of 1 and 145.2 μg of 2, 600 μg of 1 and 290.4 μg of 2, 700 μg of 1 and 18.2 μg of 2, 700 μg of 1 and 36.3 μg of 2, 700 μg of 1 and 72.6 μg of 2, 700 μg of 1 and 108.9 μg of 2, 700 μg of 1 and 145.2 μg of 2, 700 μg of 1 and 290.4 μg of 2, 800 μg of 1 and 18.2 μg of 2, 800 μg of 1 and 36.3 μg of 2, 800 μg of 1 and 72.6 μg of 2, 800 μg of 1 and 108.9 μg of 2, 800 μg of 1 and 145.2 μg of 2, 800 μg of 1 and 290.4 μg of 2, 900 μg of 1 and 18.2 μg of 2, 900 μg of 1 and 36.3 μg of 2, 900 μg of 1 and 72.6 μg of 2, 900 μg of 1 and 108.9 μg of 2, 900 μg of 1 and 145.2 μg of 2, 900 μg of 1 and 290.4 μg of 2, 1000 μg of 1 and 18.2 μg of 2, 1000 μg of 1 and 36.3 μg of 2, 1000 μg of 1 and 72.6 μg of 2, 1000 μg of 1 and 108.9 μg of 2, 1000 μg of 1 and 145.2 μg of 2, 1000 μg of 1 and 290.4 μg of 2.

The aforementioned examples of possible doses applicable for the combinations according to the invention are to be understood as referring to doses per single application. However, these examples are not be understood as excluding the possibility of administering the combinations according to the invention multiple times. Depending on the medical need, patients may receive also multiple inhalative applications. As an example, patients may receive the combinations according to the invention for instance two or three times (e.g., two or three puffs with a powder inhaler, an MDI, etc.) in the morning of each treatment day. As the aforementioned dose examples are only to be understood as dose examples per single application (i.e., per puff) multiple application of the combinations according to the invention leads to multiple doses of the aforementioned examples. The application of the compositions according to the invention can be, for instance, once a day, or, depending on the duration of action of the anticholinergic agent, twice a day, or once every 2 or 3 days.

Moreover, it is emphasized that the aforementioned dose examples are to be understood as examples of metered doses only. In other terms, the aforementioned dose examples are not to be understood as the effective doses of the combinations according to the invention that do, in fact, reach the lung. It is clear for the person of ordinary skill in the art that the delivered dose to the lung is generally lower than the metered dose of the administered active ingredients.

The active substance combinations of 1 and 2 according to the invention are preferably administered by inhalation. For this purpose, ingredients 1 and 2 have to be made available in forms suitable for inhalation. Inhalable preparations according to the invention include inhalable powders, propellant-containing metered dose aerosols, or propellant-free inhalable solutions. Inhalable powders according to the invention containing the combination of active substances 1 and 2 may consist of the active substances on their own or of a mixture of the active substances with physiologically acceptable excipients. Within the scope of the present invention, the term carrier may optionally be used instead of the term excipient. Within the scope of the present invention, the term propellant-free inhalable solutions also includes concentrates or sterile inhalable solutions ready for use. The preparations according to the invention may contain the combination of active substances 1 and 2 either together in one formulation or in two separate formulations. These formulations which may be used within the scope of the present invention are described in more detail in the next part of the specification.

### A. Inhalable Powder Containing the Combinations of Active Substances 1 and 2 According to the Invention

The inhalable powders according to the invention may contain 1 and 2 either on their own or in admixture with suitable physiologically acceptable excipients. If the active substances 1 and 2 are present in admixture with physiologically acceptable excipients, the following physiologically acceptable excipients may be used to prepare these inhalable powders according to the invention: monosaccharides (e.g., glucose or arabinose), disaccharides (e.g., lactose, saccharose, maltose, or trehalose), oligo- and polysaccharides (e.g., dextran), polyalcohols (e.g., sorbitol, mannitol, or xylitol), cyclodextrins (e.g., α-cyclodextrin, β-cyclodextrin, χ-cyclodextrin, methyl-β-cyclodextrin, hydroxypropyl-β-cyclodextrin), salts (e.g., sodium chloride or calcium carbonate) or mixtures of these excipients with one another. Preferably, mono- or disaccharides are used, while the use of lactose, trehalose, or glucose is preferred, particularly, but not exclusively, in the form of their hydrates.

Within the scope of the inhalable powders according to the invention the excipients have a maximum average particle size of up to 250 μm, preferably between 10 μm and 150 μm, most preferably between 15 μm and 80 μm. It may sometimes seem appropriate to add finer excipient fractions with an average particle size of 1 μm to 9 μm to the excipients mentioned above. These finer excipients are also selected from the group of possible excipients listed hereinbefore. Finally, in order to prepare the inhalable powders according to the invention, micronized active substance 1 and 2, preferably with an average particle size of 0.5 μm to 10 μm, more preferably from 1 μm to 6 μm, is added to the excipient mixture. Processes for producing the inhalable powders according to the invention by grinding and micronizing and by finally mixing the ingredients together are known from the prior art. The inhalable powders according to the invention may be prepared and administered either in the form of a single powder mixture which contains both 1 and 2 or in the form of separate inhalable powders which contain only 1 or 2.

The inhalable powders according to the invention may be administered using inhalers known from the prior art. Inhalable powders according to the invention which contain a physiologically acceptable excipient in addition to 1 and 2 may be administered, for example, by means of inhalers which deliver a single dose from a supply using a measuring chamber as described in U.S. Pat. No. 4,570,630, which is hereby incorporated by reference, or by other means as described in DE 36 25 685 A. The inhalable powders according to the invention which contain 1 and 2 optionally in conjunction with a physiologically acceptable excipient may be administered for example using an inhaler known by the name TURBUHALER® or using inhalers as disclosed, for example, in EP 237507 A. Preferably, the inhalable powders according to the invention which contain physiologically acceptable excipient in addition to 1 and 2 are packed into capsules (to produce so-called inhalettes) which are used in inhalers as described, for example, in WO 94/28958 (corresponding to U.S. Pat. No. 5,947,118, which is hereby incorporated by reference).

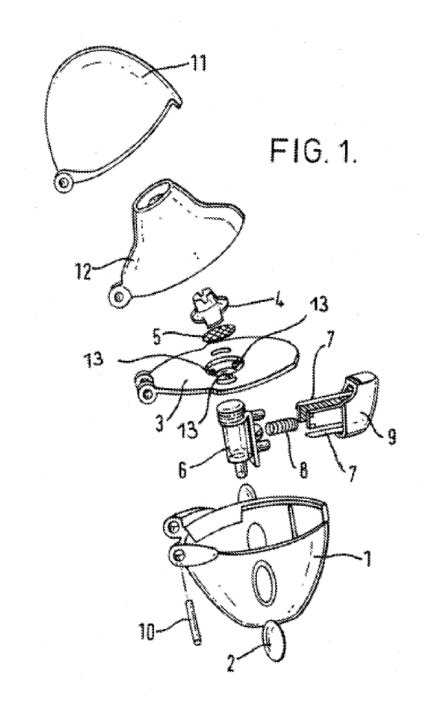

A particularly preferred inhaler for administering the pharmaceutical combination according to the invention in inhalettes is shown in FIG. 1.

The inhaler according to FIG. 1 is characterized by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, a mouthpiece 12 which is connected to the housing 1, the deck 3 and a cover 11 via a spindle 10 to enable it to be flipped open or shut and three holes 13 with diameters below 1 mm in the central region around the capsule chamber 6 and underneath the screen housing 4 and screen 5.

The main air flow enters the inhaler between deck 3 and base 1 near to the hinge. The deck has in this range a reduced width, which forms the entrance slit for the air. Then the flow reverses and enters the capsule chamber 6 through the inlet tube. The flow is then further conducted through the filter and filter holder to the mouthpiece. A small portion of the flow enters the device between mouthpiece and deck and flows then between filter holder and deck into the main stream. Due to production tolerances, there is some uncertainty in this flow because of the actual width of the slit between filter holder and deck. In case of new or reworked tools, the flow resistance of the inhaler may therefore be a little off the target value. To correct this deviation, the deck has in the central region around the capsule chamber 6 and underneath the screen housing 4 and screen 5 three holes 13 with diameters below 1 mm. Through these holes 13 flows air from the base into the main air stream and reduces such slightly the flow resistance of the inhaler. The actual diameter of these holes 13 can be chosen by proper inserts in the tools so that the mean flow resistance can be made equal to the target value.

If the inhalable powders according to the invention are packed into capsules (inhalettes) for the preferred use described above, the quantities packed into each capsule should be 1 mg to 30 mg per capsule. These capsules contain, according to the invention, either together or separately, the doses of 1 or 1′ and 2 or 2′ mentioned hereinbefore for each single dose.

### B. Propellant Gas-Driven Inhalation Aerosols Containing the Combinations of Active Substances 1 and 2

Inhalation aerosols containing propellant gas according to the invention may contain substances 1 and 2 dissolved in the propellant gas or in dispersed form. 1 and 2 may be present in separate formulations or in a single preparation, in which 1 and 2 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane, or isobutane and halohydrocarbons such as fluorinated derivatives of methane, ethane, propane, butane, cyclopropane, or cyclobutane. The propellant gases mentioned above may be used on their own or in mixtures thereof. Particularly preferred propellant gases are halogenated alkane derivatives selected from TG11, TG12, TG134a (1,1,1,2-tetrafluoroethane), and TG227 (1,1,1,2,3,3,3-heptafluoropropane), and mixtures thereof, of which the propellant gases TG134a, TG227, and mixtures thereof are preferred.

The propellant-driven inhalation aerosols according to the invention may also contain other ingredients such as cosolvents, stabilizers, surfactants, antioxidants, lubricants, and pH adjusters. All these ingredients are known in the art.

The inhalation aerosols containing propellant gas according to the invention may contain up to 5 wt.-% of active substance 1 and/or 2. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-%, or 0.5 to 1 wt.-% of active substance 1 and/or 2.

If the active substances 1 and/or 2 are present in dispersed form, the particles of active substance preferably have an average particle size of up to 10 μm, preferably from 0.1 μm to 6 μm, more preferably from 1 μm to 5 μm.

The propellant-driven inhalation aerosols according to the invention mentioned above may be administered using metered dose inhalers (MDIs) known in the art.

Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterized in that they contain the propellant gas-containing aerosols described above according to the invention. The present invention also relates to cartridges fitted with a suitable valve which can be used in a suitable inhaler and which contain one of the abovementioned propellant gas-containing inhalation aerosols according to the invention. Suitable cartridges and methods of filling these cartridges with the inhalable aerosols containing propellant gas according to the invention are known from the prior art.

### C. Propellant-Free Inhalable Solutions or Suspensions Containing the Combinations of Active Substances 1 and 2 According to the Invention

Propellant-free inhalable solutions and suspensions according to the invention contain, for example, aqueous or alcoholic, preferably ethanolic solvents, optionally ethanolic solvents mixed with aqueous solvents. If aqueous/ethanolic solvent mixtures are used the relative proportion of ethanol compared with water is not limited but preferably the maximum is up to 70 percent by volume, more particularly up to 60 percent by volume of ethanol. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulfuric acid, and/or phosphoric acid. Examples of particularly suitable organic acids include ascorbic acid, citric acid, malic acid, tartaric acid, maleic acid, succinic acid, fumaric acid, acetic acid, formic acid, and/or propionic acid etc. Preferred inorganic acids are hydrochloric and sulfuric acids. It is also possible to use the acids which have already formed an acid addition salt with one of the active substances. Of the organic acids, ascorbic acid, fumaric acid, and citric acid are preferred. If desired, mixtures of the above acids may be used, particularly in the case of acids which have other properties in addition to their acidifying qualities, e.g., as flavorings, antioxidants, or complexing agents, such as citric acid or ascorbic acid, for example. According to the invention, it is particularly preferred to use hydrochloric acid to adjust the pH.

According to the invention, the addition of edetic acid (EDTA) or one of the known salts thereof, sodium edetate, as stabilizer or complexing agent is unnecessary in the present formulation. Other embodiments may contain this compound or these compounds. In a preferred embodiment the content based on sodium edetate is less than 100 mg/100 mL, preferably less than 50 mg/100 mL, more preferably less than 20 mg/100 mL. Generally, inhalable solutions in which the content of sodium edetate is from 0 to 10 mg/100 mL are preferred.

Cosolvents and/or other excipients may be added to the propellant-free inhalable solutions which may be used according to the invention. Preferred cosolvents are those which contain hydroxyl groups or other polar groups, e.g., alcohols, particularly isopropyl alcohol, glycols, particularly propyleneglycol, polyethyleneglycol, polypropylene glycol, glycol ether, and glycerol, and polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilizers, complexing agents, antioxidants, and/or preservatives which guarantee or prolong the shelf life of the finished pharmaceutical formulation, flavorings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols, and similar vitamins and provitamins occurring in the human body.

Preservatives may be used to protect the formulation from contamination with pathogens. Suitable preservatives are those which are known in the art, particularly cetyl pyridinium chloride, benzalkonium chloride, or benzoic acid or benzoates such as sodium benzoate in the concentration known from the prior art. The preservatives mentioned above are preferably present in concentrations of up to 50 mg/100 mL, more preferably between 5 and 20 mg/100 mL.

Preferred formulations contain, in addition to the solvent water and the combination of active substances 1 and 2, only benzalkonium chloride and sodium edetate. In another preferred embodiment, no sodium edetate is present.

The propellant-free inhalable solutions which may be used within the scope of the invention are administered in particular using inhalers of the kind which are capable of nebulizing a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 10 μL and 30 μL of active substance solution can be nebulized in preferably one spray action to form an aerosol with an average particle size of less than 20 μm, preferably less than 10 μm, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 (corresponding to U.S. Pat. No. 5,497,944, which is hereby incorporated by reference) and also in WO 97/12687 (corresponding to U.S. Pat. No. 5,964,416, which is hereby incorporated by reference) (cf. in particular FIGS. 6a and 6b). The nebulizers (devices) described therein are also known by the name RESPIMAT®.

This RESPIMAT® nebulizer can advantageously be used to produce the inhalable aerosols according to the invention containing the combination of the active substances 1 and 2. Because of its cylindrical shape and handy size of less than 9 cm to 15 cm long and 2 cm to 4 cm wide, this device can be carried at all times by the patient. The nebulizer sprays a defined volume of pharmaceutical formulation using high pressures through small nozzles so as to produce inhalable aerosols.

The preferred atomizer essentially consists of an upper housing part, a pump housing, a nozzle, a locking mechanism, a spring housing, a spring, and a storage container, characterized by:
a pump housing which is secured in the upper housing part and which comprises at one end a nozzle body with the nozzle or nozzle arrangement,
a hollow plunger with valve body,
a power takeoff flange in which the hollow plunger is secured and which is located in the upper housing part,
a locking mechanism situated in the upper housing part,
a spring housing with the spring contained therein, which is rotatably mounted on the upper housing part by means of a rotary bearing, and
a lower housing part which is fitted onto the spring housing in the axial direction.

The hollow plunger with valve body corresponds to a device disclosed in WO 97/12687 (corresponding to U.S. Pat. No. 5,964,416). It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 MPa to 60 MPa (about 50 bar to 600 bar), preferably 10 MPa to 60 MPa (about 100 bar to 600 bar) on the fluid, the measured amount of active substance solution, at its high pressure end at the moment when the spring is actuated. Volumes of 10 to 50 microliters are preferred, while volumes of 10 to 20 microliters are particularly preferred and a volume of 15 microliters per spray is most particularly preferred.

The valve body is preferably mounted at the end of the hollow plunger facing the valve body.

The nozzle in the nozzle body is preferably microstructured, i.e. produced by microtechnology. Microstructured valve bodies are disclosed, for example, in WO 94/07607 (corresponding to U.S. Pat. No. 5,911,851, which is hereby incorporated by reference); reference is hereby made to the contents of this specification, particularly FIG. 1 therein and the associated description.

The nozzle body consists, for example, of two sheets of glass and/or silicon firmly joined together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns while the length is preferably 7 to 9 microns.

In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be at an angle of 20° to 160° to one another, preferably 60° to 150°, most preferably 80° to 100°. The nozzle openings are preferably arranged at a spacing of 10 to 200 microns, more preferably at a spacing of 10 to 100 microns, most preferably 30 to 70 microns. Spacings of 50 microns are most preferred. The directions of spraying will therefore meet in the vicinity of the nozzle openings.

The liquid pharmaceutical preparation strikes the nozzle body with an entry pressure of up to 600 bar, preferably 200 bar to 300 bar, and is atomized into an inhalable aerosol through the nozzle openings. The preferred particle or droplet sizes of the aerosol are up to 20 microns, preferably 3 to 10 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g. a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomizer axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomizer; this causes the deformable ring to deform in the annual plane. Details of the construction of the locking mechanism are given in WO 97/20590 (corresponding to U.S. Pat. No. 6,453,795, which is hereby incorporated by reference).

The lower housing part is pushed axially over the spring housing and covers the mounting, the drive of the spindle and the storage container for the fluid.

When the atomizer is actuated, the upper housing part is rotated relative to the lower housing part, the lower housing part taking the spring housing with it. The spring is thereby compressed and biased by means of the helical thrust gear and the locking mechanism engages automatically. The angle of rotation is preferably a whole-number fraction of 360°, e.g., 180°. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomized may be pushed into the atomizer one after another and used in succession. The storage container contains the aqueous aerosol preparation according to the invention.

The atomizing process is initiated by pressing gently on the actuating button. As a result, the locking mechanism opens up the path for the power takeoff member. The biased spring pushes the plunger into the cylinder of the pump housing. The fluid leaves the nozzle of the atomizer in atomized form.

Further details of construction are disclosed in PCT Applications WO 97/12683 (corresponding to U.S. Pat. No. 6,176,442, which is hereby incorporated by reference) and WO 97/20590 (corresponding to U.S. Pat. No. 6,176,442), to which reference is hereby made.

The components of the atomizer (nebulizer) are made of a material which is suitable for its purpose. The housing of the atomizer and, if its operation permits, other parts as well are preferably made of plastics, e.g., by injection molding. For medicinal purposes, physiologically safe materials are used.

FIGS. 6a/b of WO 97/12687 show the RESPIMAT® nebulizer which can advantageously be used for inhaling the aqueous aerosol preparations according to the invention.

FIG. 6a (WO 97/12687) shows a longitudinal section through the atomizer with the spring biased while FIG. 6b (WO 97/12687) shows a longitudinal section through the atomizer with the spring relaxed.

The upper housing part (51) contains the pump housing (52) on the end of which is mounted the holder (53) for the atomizer nozzle. In the holder is the nozzle body (54) and a filter (55). The hollow plunger (57) fixed in the power takeoff flange (56) of the locking mechanism projects partially into the cylinder of the pump housing. At its end, the hollow plunger carries the valve body (58). The hollow plunger is sealed off by means of the seal (59). Inside the upper housing part is the stop (60) on which the power takeoff flange abuts when the spring is relaxed. On the power takeoff flange is the stop (61) on which the power takeoff flange abuts when the spring is biased. After the biasing of the spring, the locking member (62) moves between the stop (61) and a support (63) in the upper housing part. The actuating button (64) is connected to the locking member. The upper housing part ends in the mouthpiece (65) and is sealed off by means of the protective cover (66) which can be placed thereon.

The spring housing (67) with compression spring (68) is rotatably mounted on the upper housing part by means of the snap-in lugs (69) and rotary bearing. The lower housing part (70) is pushed over the spring housing. Inside the spring housing is the exchangeable storage container (71) for the fluid (72) which is to be atomized. The storage container is sealed off by the stopper (73) through which the hollow plunger projects into the storage container and is immersed at its end in the fluid (supply of active substance solution). The spindle (74) for the mechanical counter is mounted in the covering of the spring housing. At the end of the spindle facing the upper housing part is the drive pinion (75). The slider (76) sits on the spindle.

The nebulizer described above is suitable for nebulizing the aerosol preparations which may be used according to the invention to produce an aerosol suitable for inhalation.

If the formulation according to the invention are nebulized using the method described above (RESPIMAT® nebulizer) the quantity delivered should correspond to a defined quantity with a tolerance of not more than 25%, preferably 20% of this amount in at least 97%, preferably at least 98% of all operations of the inhaler (spray actuations). Preferably, between 5 mg and 30 mg of formulation, most preferably between 5 mg and 20 mg of formulation are delivered as a defined mass on each actuation.

However, the formulation according to the invention may also be nebulized by means of inhalers other than those described above, e.g., jet stream inhalers or other stationary nebulizers.

Accordingly, in a further aspect, the invention relates to the method according to the invention administering pharmaceutical formulations in the form of propellant-free inhalable solutions or suspensions as described above combined with a device suitable for administering these formulations, preferably in conjunction with the RESPIMAT® nebulizer. Preferably, the invention relates to propellant-free inhalable solutions or suspensions characterized by the combination of active substances 1 and 2 according to the invention in conjunction with the RESPIMAT® nebulizer. In addition, the present invention relates to the use according to the invention of the above-mentioned devices for inhalation, preferably the RESPIMAT® nebulizer, characterized in that they contain the propellant-free inhalable solutions or suspensions according to the invention as described hereinbefore.

According to the invention, inhalable solutions which contain the active substances 1 and 2 in a single preparation are preferred. The term “single preparation” also includes preparations which contain the two ingredients 1 and 2 in two-chamber cartridges, as disclosed, for example, in WO 00/23037 (corresponding to U.S. Pat. No. 6,481,435, which is hereby incorporated by reference).

The propellant-free inhalable solutions or suspensions which may be used within the scope of the invention may take the form of concentrates or sterile inhalable solutions or suspensions ready for use, as well as the abovementioned solutions and suspensions designed for use in a RESPIMAT® nebulizer. Formulations ready for use may be produced from the concentrates, for example, by the addition of isotonic saline solutions. Sterile formulations ready for use may be administered using energy-operated fixed or portable nebulizers which produce inhalable aerosols by means of ultrasound or compressed air by the Venturi principle or other principles.

Accordingly, in another aspect, the present invention relates to pharmaceutical compositions in the form of propellant-free inhalable solutions or suspensions as described hereinbefore which take the form of concentrates or sterile formulations ready for use, combined with a device suitable for administering these solutions, characterized in that the device is an energy-operated free-standing or portable nebulizer which produces inhalable aerosols by means of ultrasound or compressed air by the Venturi principle or other methods.

The Examples which follow serve to illustrate the present invention in more detail without restricting the scope of the invention to the following embodiments by way of example.

### Examples of Formulations

The following examples of formulations, which may be obtained analogously to methods known in the art, serve to illustrate the present invention more fully without restricting it to the contents of these examples. In the following examples the enantiomerically pure form of the preferred enantiomer of the bromide of formula 1 (i.e., formula 1-en) is used as the active ingredient.

Inhalable Powders

| | |
|---|---|
| Ingredients | μg per capsule |

1)

| | |
|---|---|
| 1-en (bromide) | 150 |
| Formoterol fumarate dihydrate | 50 |
| Lactose | 12300 |
| Total | 12500 |

2)

| | |
|---|---|
| 1-en (bromide) | 150 |
| Salmeterol xinafoate | 50 |
| Lactose | 12300 |
| Total | 12500 |

3)

| | |
|---|---|
| 1-en (bromide) | 150 |
| Salmeterol xinafoate | 150 |
| Lactose | 12200 |
| Total | 12500 |

4)

| | |
|---|---|
| 1-en (bromide) | 200 |
| Formoterol fumarate dihydrate | 50 |
| Lactose | 24750 |
| Total | 25000 |

B) Propellant-Containing Inhalable Aerosols

| | |
|---|---|
| Ingredients | % by weight |

1) Suspension Aerosol

| | |
|---|---|
| 1-en (bromide) | 0.050 |
| Salmeterol xinafoate | 0.066 |
| Soya lecithin | 0.2 |
| TG 134a:TG 227 (2:3) | ad 100 |

2) Suspension Aerosol

| | |
|---|---|
| 1-en (bromide) | 0.080 |
| Salmeterol xinafoate | 0.033 |
| Absolute ethanol | 0.5 |
| Isopropyl myristate | 0.1 |
| TG 227 | ad 100 |

3) Suspension Aerosol

| | |
|---|---|
| 1-en (bromide) | 0.050 |
| Formoterol fumarate dihydrate | 0.035 |
| Soya lecithin | 0.2 |
| TG 134a:TG 227 (2:3) | ad 100 |

1. A pharmaceutical composition comprising:
(a) a compound of formula 1 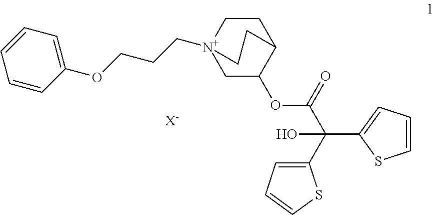
or an enantiomer, mixture of enantiomers, or a racemate thereof, wherein X⁻ is an anion with a single negative charge; and
(b) formoterol,
or an enantiomer, mixture of enantiomers, racemate or salt thereof.

2. The pharmaceutical composition of claim 1, further comprising a pharmaceutically acceptable excipient.

3. The pharmaceutical composition of claim 1, wherein X⁻ is fluoride, chloride, bromide, iodide, sulfate, phosphate, methanesulfonate, nitrate, maleate, acetate, citrate, fumarate, tartrate, oxalate, succinate, benzoate, or p-toluenesulfonate.

4. (canceled)

5. The pharmaceutical composition of claim 1, wherein the formoterol is a formoterol salt selected from sodium salts, sulfobenzoates, phosphates, isonicotinates, acetates, propionates, dihydrogen phosphates, palmitates, pivalates, or furoates, or mixtures thereof.

6. The pharmaceutical composition of claim 1, wherein formoterol is a formoterol salt selected from hydrochloride, sulfate or fumarate.

7. (canceled)

8. The pharmaceutical composition of claim 1, wherein the weight ratio of the compound of formula 1 to formoterol are in a range from about 1:5 to 500:1.

9. The pharmaceutical composition according to claim 1, wherein the pharmaceutical composition is suitable for inhalation.

10. The pharmaceutical composition according to claim 1, wherein the compound of formula 1 is an enantiomer of formula 1-en

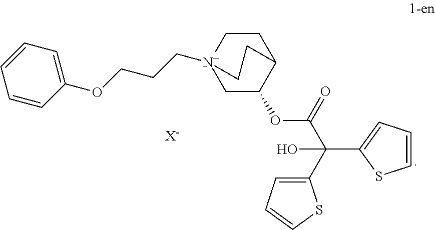

11. The pharmaceutical composition according to claim 9, wherein the compound of formula 1 is an enantiomer of formula 1-en

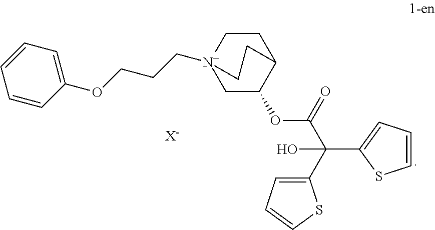

12. The pharmaceutical composition according to claim 9, wherein the pharmaceutical composition is an inhalable powder, a propellant-containing metering aerosol, or a propellant-free inhalable solution or suspension.

13. (canceled)

14. The pharmaceutical composition according to claim 2, wherein the pharmaceutical composition further comprises a suitable physiologically acceptable excipient selected from the group consisting of: monosaccharides, disaccharides, oligo- and polysaccharides, polyalcohols, and salts.

15. (canceled)

16. The pharmaceutical composition of claim 14, wherein the excipient has a maximum average particle size of up to 250 μm.

17. (canceled)

18. The pharmaceutical composition of claim 16, wherein the excipient has a maximum average particle size of between 10 μm and 150 μm.

19. A capsule containing a pharmaceutical composition according to claim 1, in the form of an inhalable powder.

20. A capsule containing a pharmaceutical composition according to claim 10 in the form of an inhalable powder.

21. A pharmaceutical composition according to claim 1, wherein the pharmaceutical composition is a propellant-containing inhalable aerosol and the compound of formula 1 and the formoterol are in dissolved or dispersed form.

22. The pharmaceutical composition according to claim 21, wherein the propellant-containing inhalable aerosol comprises a propellant gas selected from hydrocarbons and halohydrocarbons.

23. The pharmaceutical composition according to claim 21, wherein the propellant-containing inhalable aerosol comprises a propellant gas selected from the group consisting of: n-propane; n-butane; isobutane; and chlorinated and/or fluorinated derivatives of methane, ethane, propane, butane, cyclopropane, and cyclobutane.

24. The pharmaceutical composition according to claim 22, wherein the propellant gas is TG134a, TG227, or a mixture thereof.

25. The pharmaceutical composition according to claim 21, further comprising at least one of a cosolvent, stabilizer, surfactant, antioxidant, lubricant, or means for adjusting the pH of the composition.

26. The pharmaceutical composition according to claim 22, further comprising at least one of a cosolvent, stabilizer, surfactant, antioxidant, lubricant, or means for adjusting the pH of the composition.

27. The pharmaceutical composition according to claim 21, wherein the amount of the compound of formula 1′ or the formoterol is up to 5 wt. % of the pharmaceutical composition.

28. A pharmaceutical composition according to claim 1, wherein the pharmaceutical composition is propellant-free inhalable solution or suspension that further comprises a solvent selected from water, ethanol, or a mixture of water and ethanol.

29. The pharmaceutical composition according to claim 28, wherein the pH is between 2 and 7.

30. The pharmaceutical composition according to claim 29, wherein the pH is between 2 and 5.

31. The pharmaceutical composition according to claim 28, wherein the pH of the pharmaceutical composition is adjusted by means of one or more acids selected from the group consisting of: hydrochloric acid, hydrobromic acid, nitric acid, sulfuric acid, ascorbic acid, citric acid, malic acid, tartaric acid, maleic acid, succinic acid, fumaric acid, acetic acid, formic acid, and propionic acid.

32. The pharmaceutical composition according to claim 28, further comprising other cosolvents or excipients.

33. The pharmaceutical composition according to claim 31, further comprising other cosolvents or excipients.

34. The pharmaceutical composition according to claim 32, wherein the composition contains a cosolvent is selected from the group consisting of alcohols, glycols, polyoxyethylene alcohols, and polyoxyethylene fatty acid esters.

35. The pharmaceutical composition according to claim 32, wherein the composition contains a cosolvent is selected from the group consisting of: isopropyl alcohol, propylene glycol, polyethylene glycol, polypropylene glycol, glycol ether, and glycerol.

36. The pharmaceutical composition according to claim 32, wherein the composition contains an excipient is selected from the group consisting of: surfactants, stabilizers, complexing agents, antioxidants, preservatives, flavorings, pharmacologically acceptable salts, and vitamins.

37. The pharmaceutical composition according to claim 36, wherein the composition contains an excipient is selected from the group consisting of: edetic acid, a salt of edetic acid, ascorbic acid, vitamin A, vitamin E, tocopherols, cetyl pyridinium chloride, benzalkonium chloride, benzoic acid, and benzoate salts.

38. A pharmaceutical composition consisting essentially of:
(a) a compound of formula 1 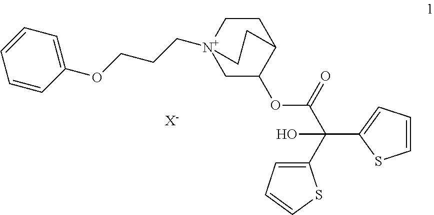
wherein X⁻ is an anion with a single negative charge;
(b) formoterol;
(c) a solvent;
(d) benzalkonium chloride; and
(e) sodium edetate,
wherein the compound of formula 1 is optionally an enantiomer, mixture of enantiomers, or a racemate thereof and the formoterol is optionally an enantiomer, mixtures of their enantiomers, racemate, or salt thereof.

39. A pharmaceutical composition consisting essentially of:
(a) a compound of formula 1 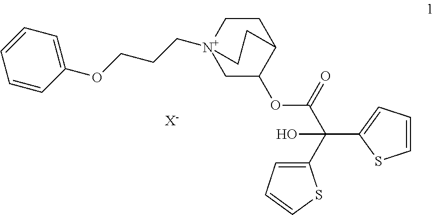
wherein X⁻ is an anion with a single negative charge;
(b) formoterol;
(c) a solvent; and
(d) benzalkonium chloride,
wherein the compound of formula 1 is optionally an enantiomer, mixture of enantiomers, or a racemate thereof and the formoterol is optionally an enantiomer, mixtures of their enantiomers, racemate, or salt thereof.

40. A pharmaceutical composition consisting essentially of:
(a) a compound of formula 1-en 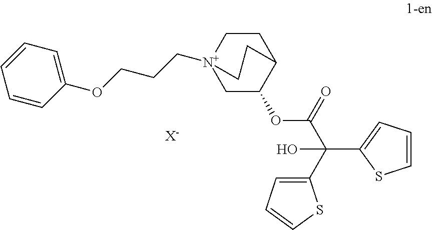
wherein X⁻ is an anion with a single negative charge;
(b) formoterol;
(c) a solvent;
(d) benzalkonium chloride; and
(e) sodium edetate,
wherein the formoterol is optionally in the form of an enantiomer, mixture of enantiomers, racemate or salt thereof.

41. A pharmaceutical composition consisting essentially of:
(a) a compound of formula 1-en 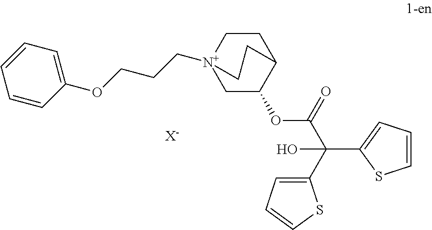
wherein X⁻ is an anion with a single negative charge;
(b) formoterol;
(c) a solvent; and
(d) benzalkonium chloride,
wherein the formoterol is optionally in the form of an enantiomer, mixture of enantiomers, racemate or salt thereof.

42. A method of treating COPD or asthma in a patient in need of such treatment, the method comprising administering to the patient a therapeutically effective amount of the pharmaceutical composition according to claim 1.

43. A kit comprising one or more unit dosage containers containing a pharmaceutical composition, each unit dosage container containing a pharmaceutical composition comprising:
(a) a compound of formula 1 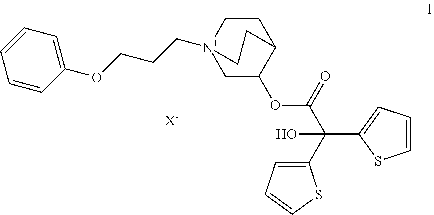
wherein X⁻ is an anion with a single negative charge; and
(b) formoterol,
each optionally together with a pharmaceutically acceptable excipient, wherein the compound of formula 1 is optionally an enantiomer, mixture of enantiomers, or a racemate thereof and the formoterol is optionally an enantiomer, mixtures of their enantiomers, racemate, or salt thereof.

44. The kit according to claim 43, further comprising instructions with directions for using the kit.

45. A kit comprising:
(a) a first container containing a first pharmaceutical formulation comprising a compound of formula 1 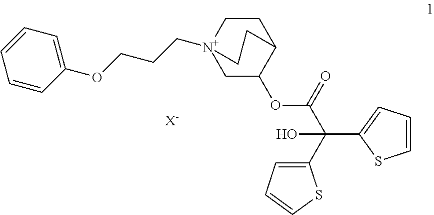
wherein X⁻ is an anion with a single negative charge; and
(b) a second container containing a second pharmaceutical formulation comprising formoterol,
each container each optionally further containing a pharmaceutically acceptable excipient, wherein the compound of formula 1 is optionally an enantiomer, mixture of enantiomers, or a racemate thereof and the formoterol is optionally an enantiomer, mixtures of their enantiomers, racemate, or salt thereof.

46. The kit according to claim 45, further comprising instructions with directions for using the kit.

47. The pharmaceutical composition of claim 1, wherein the component (b) is a formoterol salt, wherein the formoterol salt is formoterol fumerate or a dihydrate of formoterol fumerate.

48. The pharmaceutical composition of claim 1, wherein X⁻ is bromide.

49. The pharmaceutical composition of claim 1, wherein the compound of formula 1 and formoterol in a ratio by weight ranging from 1:5 to 500:1.

50. The pharmaceutical composition of claim 1, wherein the compound of formula 1 and formoterol in a ratio by weight ranging from 1:5 to 200:1.

51. The pharmaceutical composition of claim 1, wherein the compound of formula 1 and formoterol in a ratio by weight ranging from 1:2 to 100:1.
